(19) United States
(12) Patent Application Publication
(10) Pub. No.: US 2024/0144629 A1
(43) Pub. Date: May 2, 2024

(54) SYSTEM AND METHOD FOR IMAGING A CONTAINER

(71) Applicant: BD KIESTRA B.V., Drachten (NL)

(72) Inventors: Franciscus Hermannus Feijen, Leeuwarden (NL); Brent Ronald Pohl, Timonium, MD (US); Craig W. Bark, Fallston, MD (US); Jan Bart Van Der Vijver, Groningen (NL); Timothy Roy Hansen, Spring Grove, PA (US); Jurjen Sinnema, Joure (NL); Martijn Kleefstra, Surhuisterveen (NL)

(73) Assignee: BD KIESTRA B.V., Drachten (NL)

(21) Appl. No.: 18/280,997

(22) PCT Filed: Mar. 9, 2022
(86) PCT No.: PCT/US2022/019424

§ 371 Date: Sep. 8, 2023

Related U.S. Application Data
(60) Provisional application No. 63/159,269, filed on Mar. 10, 2021.

(51) Int. Cl. G06V 10/14; G06V 10/22; G06V 10/25

(52) U.S. Cl. CPC G06V 10/14; G06V 10/22; G06V 10/25; G06V 2201/034

(57) ABSTRACT

A system that may automatically and rapidly process blood culture bottles. Such processing includes obtaining an image of a label placed on the cylindrical surface of the blood culture bottle. The system may also determine an amount of blood sample that has been added to the blood culture bottle by comparing a sample level to pre-placed fiducial. The distance between the liquid meniscus in the blood culture bottle and fiducial may be used to determine blood volume. The imaging apparatus may also detect internal conditions of the blood culture bottle such as the presence of foam or the presence of culture media in a neck portion of the blood culture bottle. The image of the label is obtained from one or more images captured by a camera or scanner. The information on the label may be read automatically and therefore the blood culture bottles do not have to be processed manually, increasing throughput of the apparatus.

### CROSS-REFERENCE TO RELATED APPLICATIONS

This application claims priority from U.S. Provisional Application Ser. No. 63/159,269 filed Mar. 10, 2021, which is incorporated by reference herein.

### TECHNICAL FIELD

Presently described is an apparatus that obtains a single image of a blood culture bottle from which information such as label information and fill level may be obtained.

### BACKGROUND

The presence of biologically active agents such as bacteria in a patient's body fluid, especially blood, is generally determined using blood culture bottles. A small quantity of blood is injected through an enclosing rubber septum into a sterile bottle containing a culture medium, and the bottle is then incubated at about 35° C. and monitored for microorganism growth. Microbial growth is detected by a change in the blood culture over time that is an indication of microbial growth. Typically, parameters such as the concentration of carbon dioxide or oxygen in the culture bottle headspace or a change in pH are monitored for changes over time that are indicative of microbial growth.

Since it is of utmost importance to learn if a patient has a bacterial infection, hospitals and laboratories have automated apparatus that may process many blood culture bottles simultaneously. One example of such an apparatus is the BD BACTEC™ system, which is manufactured and sold by Becton, Dickinson and Co. U.S. Pat. No. 5,817,508 to Berndt et al. describes a prior art blood culture apparatus, and is incorporated by reference herein. Additional descriptions of Blood Culture Apparatus are provided in U.S. Pat. No. 5,516,692 (“Compact Blood Culture Apparatus”) and U.S. Pat. No. 5,498,543 (“Sub-Compact Blood Culture Apparatus”) both of which are incorporated by reference herein.

It is critical to ensure that the presence or absence of a blood stream infection (BSI) is correctly determined. Patients and their caregivers are placed at risk if a BSI goes undetected. It is well known that overfilling a blood culture bottle with the blood sample may lead to false positives. It is well known that underfilling blood culture bottles with the blood sample may lead to false negatives. This is because the sample removed from the patient has a certain, but unknown, concentration of bacteria (if bacteria is at all present). Therefore, in the case of underfill, a lower bacteria count is present in the blood culture bottle at time zero than if the culture bottle had been filled with the target sample amount. It follows then that, in the case of overfill, a higher bacteria count is present in the blood culture bottle at time zero than if the culture bottle had been filled with the target sample (e.g., blood) amount. If a bottle is underfilled or overfilled, algorithms may be applied to the measured changes in carbon dioxide or oxygen concentration or pH to adjust for underfill or overfill. If the underfill or overfill exceeds a certain specification, the blood culture bottles are discarded. This is described in U.S. Pat. No. 9,365,814 which issued on Jun. 14, 2016 and is incorporated by reference herein.

Therefore, when processing blood culture bottles in a laboratory environment that is processing a large number of blood culture bottles, there is a need to be able to monitor the fill condition of each bottle accurately. Other information about the blood culture, such as the label information, is also collected. Consequently, methods and apparatus that may accurately obtain fill information and label information from a blood culture bottle continue to be sought.

### BRIEF SUMMARY

In blood culture instrumentation it is beneficial to identify the amount of blood sample that has been inoculated into the sample container (e.g., a blood culture bottle). In the context of blood cultures, the amount of sample is directly proportional to the likelihood of obtaining a bacterial colony which would subsequently be incubated, grown, and detected. In general, it is advantageous for a user (i.e., an operator or technician or phlebotomist) to ensure that the amount of blood being collected is as close as possible to the intended fill level. Underfilling the culture bottle with sample could result in not collecting a colony forming unit and therefore not obtaining a correct result for the patient.

Described herein are systems, apparatus, controls and methods to accurately and precisely determine the volume of sample inoculated into the container. Described herein is an apparatus that determines the volume of sample (e.g., blood) inoculated into the container (e.g., a blood culture bottle) by obtaining an image of the container inoculated with sample. Such an approach facilitates automation, as an operator does not need to visually inspect each bottle.

One aspect of the system described herein is a positioning apparatus that places the sample container in position for imaging.

Another aspect of the system described herein is imaging apparatus that may obtain a readable image of the blood culture bottle, which is round. Because such labels carry a bar code, the images are readable by machine vision. Because the image of the label is used to obtain information about the contents of the bottle, the image must be readable by an operator. This requires rendering a “flat” image from a curved label.

Another aspect of the system described herein is conveying and placing the sample for downstream processing after an image of the label is obtained.

Another aspect of the system is the ability to provide fast throughput of multiple sample containers, not limited to sample containers of the same or similar size. Using the imaging systems and methods described herein, other sample container conditions, (i.e., fill level, foam, media beads in the neck of the sample bottle, the presence of clotted blood in the neck of the culture bottle), are also detected.

### BRIEF DESCRIPTION OF DRAWINGS

The foregoing and other objects and advantages will be apparent upon consideration of the following detailed description, taken in conjunction with the accompanying drawings, in which like reference characters refer to like parts throughout and in which:

FIG. 1A is a schematic side view of a system for obtaining an image of a blood culture bottle;

FIG. 1B is a schematic top view of the system illustrated in FIG. 1A;

FIG. 2 is a schematic view of one embodiment of the system described herein;

FIG. 3 is a schematic view of an alternative embodiment of the system described herein;

FIG. 4A is a blood culture bottle that is placed in the system described herein to obtain an image thereof;

FIG. 4B is an image of the blood culture bottle illustrated in FIG. 4A, the image having been obtained using a system as illustrated in FIG. 2;

FIG. 4C is a polar transform of the image illustrated in FIG. 4B;

FIG. 5A-FIG. 5C are alternate configurations of AMM configurations;

FIG. 6 is a schematic view of an alternative embodiment of the system described herein; and

FIG. 7 is a schematic view of an alternative embodiment of the system described herein.

FIG. 8A-FIG. 8C are different perspective views of a conical mirror imaging module according to one embodiment described herein.

FIG. 9A and FIG. 9B are bottom views of the bracket illustrated in FIGS. 8A-8C.

FIG. 10A-FIG. 10D are perspective and side views of a conical mirror imaging module according to a second embodiment described herein.

FIG. 11 illustrates an alternate embodiment of a conical mirror imaging module with a trap door for removing the sample container from the conical mirror imaging position.

FIG. 12 illustrates and alternate imaging apparatus for the system described herein that uses a scanner and rotating platform to obtain an image of the label and a robotic gripper for placing the bottle vertically and removing the bottle horizontally.

FIG. 13 is a bottom perspective exploded view of the apparatus of FIG. 12.

FIG. 14A-FIG. 14D are perspective views of an alternative embodiment of the apparatus described herein wherein a gate is operated from beneath a platform to dispense a cylindrical sample container such as illustrated in FIG. 13 when an image of the label on the cylindrical sample bottle has been obtained.

FIG. 15 is a schematic perspective view of the imaging apparatus of FIG. 12 using the conical mirrored tray illustrated in FIG. 11.

FIG. 16 is a schematic perspective view of the imaging apparatus of FIG. 15 using multiple cameras to obtain images of discrete portions of the label.

FIG. 17 is a schematic perspective view of the imaging apparatus of FIG. 15 without the trap door.

FIG. 18 is a schematic perspective view of the imaging apparatus of FIG. 12 without the trap door.

FIG. 19 is a schematic perspective view of the imaging apparatus of FIG. 18 with an alternative configuration for gripping the sample container.

FIG. 20 is a schematic perspective view of a chute for removing the sample container from the imaging apparatus after imaging.

FIG. 21 is a flow chart of a method described herein.

### DETAILED DESCRIPTION

Aspects of the present disclosure are described in detail with reference to the drawing figures wherein like reference numerals identify similar or identical elements. It is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Described herein is an imaging system for obtaining an image of a blood culture bottle that may be used to obtain information such as label information, fill level, etc. In one particular aspect, described herein is an apparatus that may obtain one single image of the entire cylindrical body of a blood culture bottle. From that image, information such as the complete label information on the bottle and the liquid height level in the bottle may be obtained.

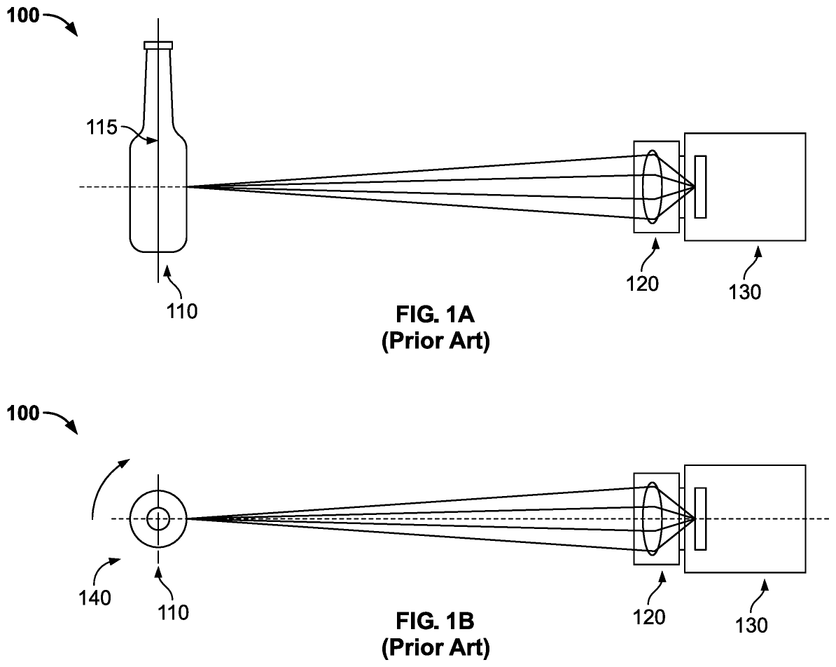

Referring to FIG. 1A, the system 100 of the prior art obtain an image of a cylindrical body that is illustrated as a blood culture bottle 110. The blood culture bottle 110 has a curved surface spanning 360° in the horizontal plane of the imaging apparatus 100. Such systems may obtain a full image of the whole cylindrical body of a blood culture bottle in one of two ways.

A simple imaging system of a lens 120 and a camera 130 obtains an image of the bottle 110. Although not shown to scale, FIG. 1 illustrates that the distance between the system and the bottle is not much greater than the length of the bottle 110. The bottle 110 is rotated around its vertical axis 115. A series of images is obtained as the bottle 110 is rotated about its axis. While the number of images might vary, one series of images for one period of a complete rotation of the bottle might number about 24 to 48 or more frames. Each image frame is communicated to an image processing apparatus to stitch together a central portion of each frame of images. From this a full image of the whole cylindrical body of the bottle is recovered. FIG. 1B is a top view of the system of FIG. 1A. The rotating platform 140 on which the bottle 110 is placed for rotation is illustrated in FIG. 1B. A system for obtaining an image of a blood culture bottle on a rotating platform is described in U.S. Pat. No. 10,395,357, which issued on Aug. 27, 2019 and is incorporated by reference herein. The image is obtained to detect the presence of foam in the containers.

In an alternate approach to that illustrated in FIGS. 1A and 1B, multiple instances of a lens/camera assembly may be positioned around the cylindrical bottle. The number of lens/camera assemblies may vary. For example, to obtain a complete image of the cylindrical bottle, twelve, sixteen, or even more lens/camera assemblies may be positioned encircling the bottle. The bottle is positioned in the center of the annular imaging zone defined by the lens/camera assemblies encircling the zone. Each lens/camera assembly obtains a discrete frame of an image of the entire bottle. The assembly then communicates the frame to an image processing module that stitches the image together using the center portion of each image frame.

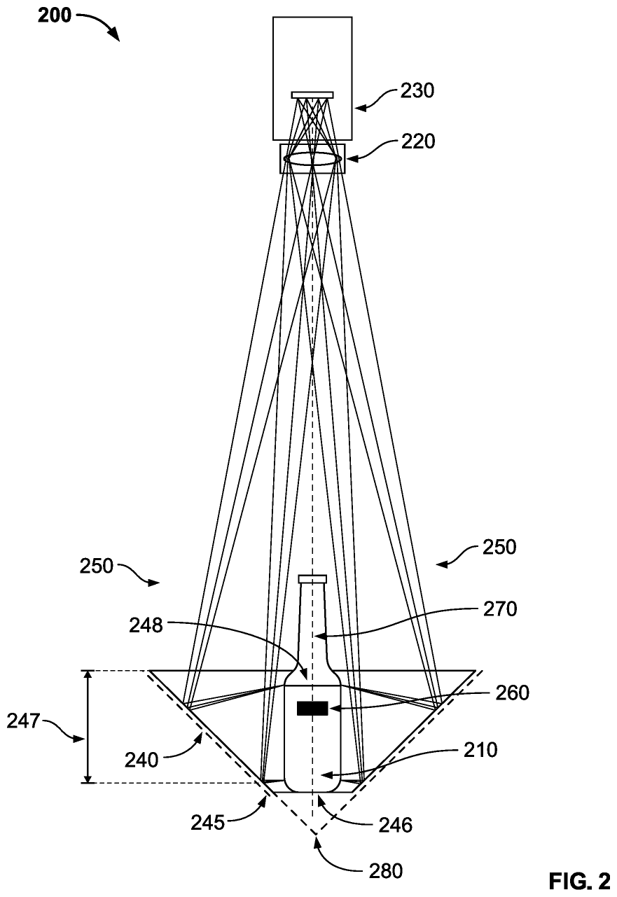

With reference to FIG. 2, the system 200 is a departure from the prior art system illustrated in FIG. 1A and FIG. 1B in that the system does not have or require a rotating platform or multiple lens/camera assemblies to obtain a 360° image of a cylindrical object such as a blood culture bottle. The system deploys what is referred to herein as an Auxiliary Mirror Module (AMM) in cooperation with a simple imaging system consisting of a lens 220 and a camera 230.

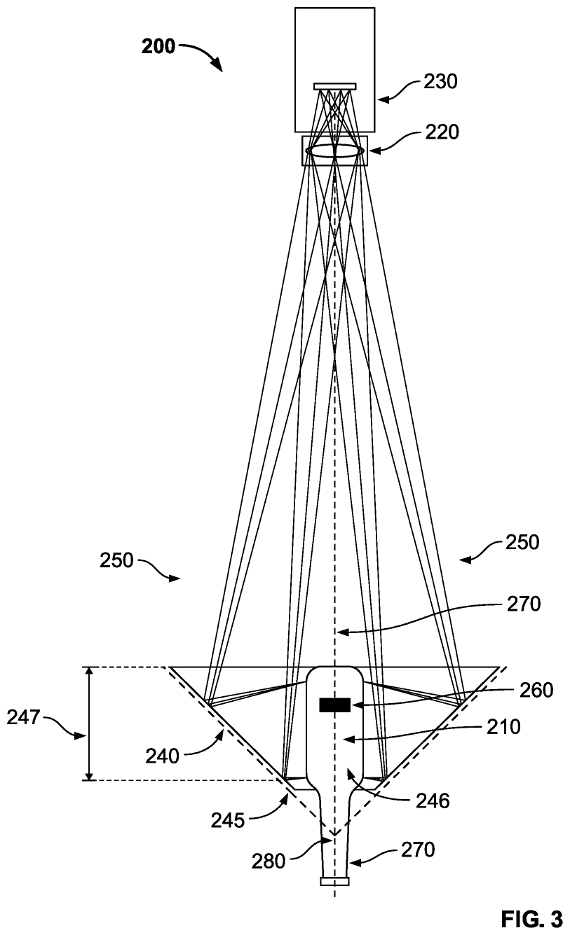

Referring again to FIG. 2, it is important to note that the bottle 210 is not required to be upright (i.e., as illustrated in FIG. 2, the bottom of the bottle 210 is proximal to the extrapolated apex 280 to measure the bottle fill or to read the label 260). The bottle 210 may be positioned on its side for imaging. Also, the bottle 210 may be positioned upside down so that the neck 270 of the bottle 210 is proximal to the apex of the AMM. The orientation of the bottle during imaging depends somewhat on the information being sought. If the objective is to obtain both the label information from the image and the fill level of the bottle, the bottle needs to be positioned upright. If the only information sought is the label image, the bottle may be positioned on its side, upside down, etc. The apex, 280, is extrapolated from the tapered sides of the AMM 240. As illustrated in FIG. 3, the bottle 210 may be positioned so that its neck 270 may be proximal to the apex of the AMM. The system 200 deploys an AMM 240 that provides for three-dimensional (3D) optical path folding. The AMM module is configured as a mirrored conical structure that reflects the bottle 210 as illustrated by rays 250. Rays 250 indicate how the bottle's reflection in the AMM is received by the lens/camera 220/230 assembly. The bottle 210 is placed at the center of the AMM and imaged by the camera through the folded path 250. By working in this specific way, an image of the entire bottle is captured in a single image frame. The image of the bottle 210 that is received by the camera sensor is a deformed image due to the nature of the bottle reflection transmitted by the AMM. However, no image stitching is required, although image processing is required to obtain a true image of the bottle from what is reflected by the AMM. The fact that no image stitching is required and no bottle rotation is required are advantages over prior art systems that obtain an image of a blood culture bottle.

Referring to FIG. 2, the AMM 240 is a special reflective mirror having a funnel shape, or conical shape, which is defined by a few parameters. The cone angle, defined at extrapolated apex 280, is 90° for the embodiment illustrated in FIG. 2. The AMM has a small circular opening 246 in a bottom 245 of the AMM that has a diameter that is slightly larger than that of the bottle 210. The AMM has a height 247 that is slightly higher than the body portion of the bottle 210. That is, most of the neck 270 of the bottle 210 extends above the AMM in the embodiment illustrated in FIG. 2. As illustrated by rays 250, the AMM provides path-folding of the reflected image of the bottle 210 in a 3D manner from every point on the portion of the bottle positioned within the AMM, to the imaging camera, forming point-to-point images. Consequently, an image of the entire bottle 210 is obtained in one frame.

Conventional mirror materials (i.e., glass with reflective backing) are contemplated. However, alternate materials of choice for spherical mirror are engineered plastics such as ABS, PC, Nylon or blends of such, polished aluminum or steel. Any of these materials may be coated to enhance reflectance using a thin layer of aluminum, beryllium, chromium, copper, gold, molybdenum, nickel, platinum, rhodium, tungsten and most commonly silver.

Referring to FIG. 3, the system is identical to that of FIG. 2 except the neck 270 of the bottle 210 is inserted through the opening 246 of the AMM. Because the bottle 210 is to be supported by an associated holding mechanism, the bottle 210 may be positioned as illustrated, which is a vertical orientation. For purposes of determining the fill level of the bottle, it is preferred to have the bottle 210 held in a bottom down vertical orientation. While the AMM would provide an image of the bottle even if the bottle was held in a horizontal position, for level sensing the measurement is more precise if the bottle is in a vertical orientation.

In one embodiment, the bottle 210 is equipped with a fill line 248 (FIG. 2). The fill line 248 serves as a reference to determine, from the image, if the bottle is precisely filled, overfilled or underfilled. In one embodiment, the fill line may be provided on the label.

The AMM described herein provides several advantages over other systems that obtain an image of a culture bottle. As noted above, there is no need to move (i.e. rotate) the bottle. For level sensing, it is advantageous if the bottle remains still for imaging. Also, only one lens/camera assembly is required, reducing the cost and complexity of the system. As noted above, only one frame is required to obtain an image of the entire bottle reducing image processing complexity. Specifically, it is less complicated to obtain a single image of a label and remediate image distortion caused by the curvature of the bottle than to stitch multiple discrete images of the label together to obtain an image of an undistorted (i.e., “flat”) label.

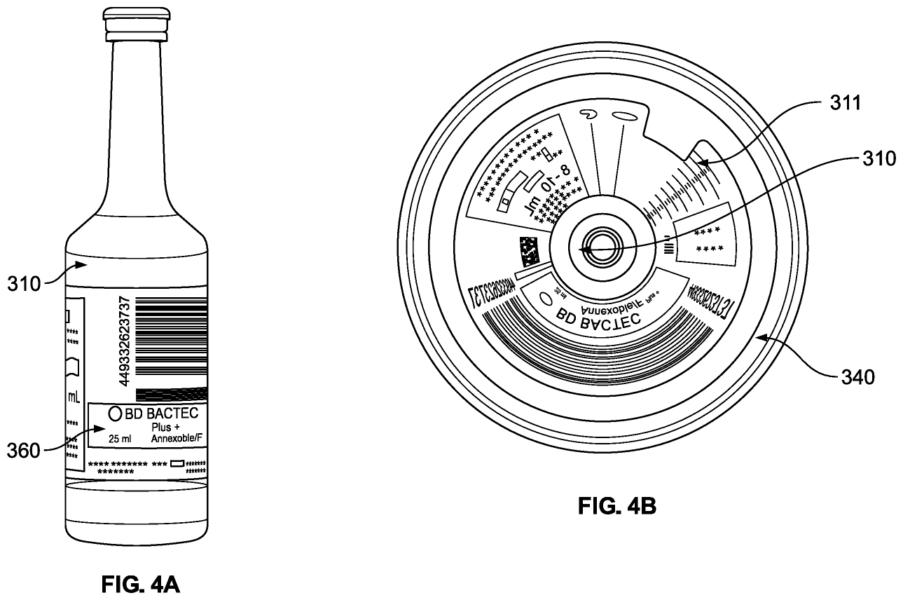

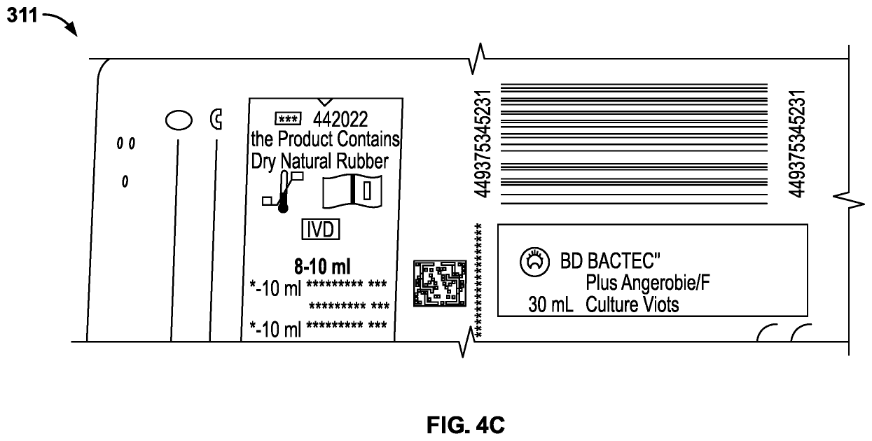

FIG. 4A is an image of a bottle 310 with the label 360 thereon. FIG. 4B illustrates an image 311 of the bottle 310 placed in the AMM 340. The AMM 340 is a mirrored conical receptacle as illustrated in FIG. 2 and FIG. 3. The bottle 310 is positioned such that the bottom of the bottle is proximal to the apex of the conical shape defined by the AMM 340. The deformed image 311 illustrated in FIG. 4B has an outer zoning region that has a higher pixel density (or resolution) than that in the inner zoning region. One way to control or to balance the Region of Interest (ROI) on the final image, is to take the image of the bottle with the neck proximal to the apex of the AMM illustrated in FIG. 3. When the neck is extending from the AMM in this manner, the neck may be held by a robot (not shown). Since the robot is positioned on the side of the AMM away from the lens/camera assembly, the robot is outside the optical path from the AMM to the lens/camera assembly.

FIG. 4C is a polar transform of the image illustrated in FIG. 4B. Techniques for forming a rectangular image from a circular image using a log-polar transform are described in U.S. Pat. No. 7,961,982 to Sibiryakov, et al., which is incorporated by reference herein. One example of suitable polar transform equations is:

X=r sin(φ) (1)

Y=r cos(φ) (2)

where r is the distance from the origin in the plane. Such techniques are well-known to one skilled in the art and not described in detail herein.

As noted above, obtaining the image of the full label in the manner described herein is advantageous because it provides all of the data regarding the label in a single data set. The full-label image is deformedly formed in an annular area for image processing as illustrated in FIG. 4B. FIG. 4C illustrates the image of the label in FIG. 4B after the application of a polar transform. Because all of the data needed to process the image information is obtained in a single frame, data acquisition is faster. As noted above, there is no need to rotate the bottle, or the imaging apparatus, to obtain multiple images of a single label. Because there is no need to move the bottle during imaging, there is no imaging error associated with mechanical noise from vibration (which may cause movement of the bottle in the y axis). Imaging errors that might arise from axial runout (i.e., wobble) are also avoided. Imaging errors could also result if the bottle moves in the radial direction between two images, which could cause label image size variation between the two images. Also, obtaining a single image of the label allows a more accurate image to be obtained of a poorly applied label (i.e., a crooked label, a wrinkled label, etc.)

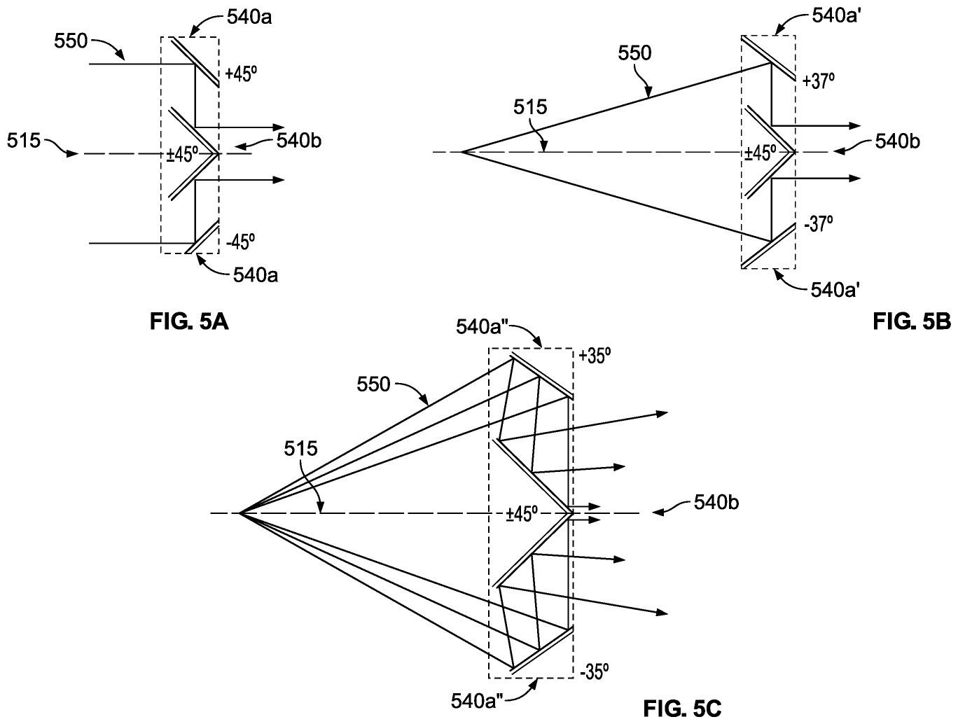

Referring to FIG. 5A-FIG. 5C, a few variations of types of AMM are illustrated. These AMMS do not provide for full bottle imaging. Rather, each of the AMMs support an enlarged field of view that captures more of the label in a single frame that may be captured in a single frame using the AMM of FIG. 1A. For example, the amount of the label obtained in a single image frame using the AMMs of FIG. 5A-FIG. 5C is about twice the amount of label obtained using an AMM does not have the modifications illustrated in FIG. 5A-FIG. 5C. The field of view is approximately doubled in the AMMs illustrated in FIG. 5A-FIG. 5C. FIG. 5A illustrates an AMM with two pairs of mirrors 540a and 540b, each of which is angled 45° relative to the horizontal line from the bottle axis 515. The optical path from the bottle (the bottle is not shown) to the lens/camera assembly (the lens/camera assembly is not shown) is illustrated by rays 550. FIG. 5B illustrates a variation of the AMM illustrated in FIG. 5A, in which the outer pair of mirrors 540a′ are positioned at a 37° angle relative to the bottle axis 515. The optical path, characterized by rays 550, illustrates a wider field of view than the AMM illustrated in FIG. 5A.

FIG. 5C illustrates another variation of the AMM illustrated in FIG. 5A and FIG. 5B, in which the outer pair of mirrors 540a′ are positioned at a 35° angle relative to the bottle axis 515. The optical path, characterized by rays 550, illustrates a wider field of view than the AMM illustrated in FIG. 5A and FIG. 5B.

Other techniques for obtaining “flat” images of bottle labels are known. Techniques that use a standard imaging device such as a camera phone or scanner are well-known and one description of such techniques is described in Slatcher, Steve, “How to create flat rectangular images of wine bottle labels,” (Feb. 21, 2018) wineous.co.uk/wp/archives/11397.

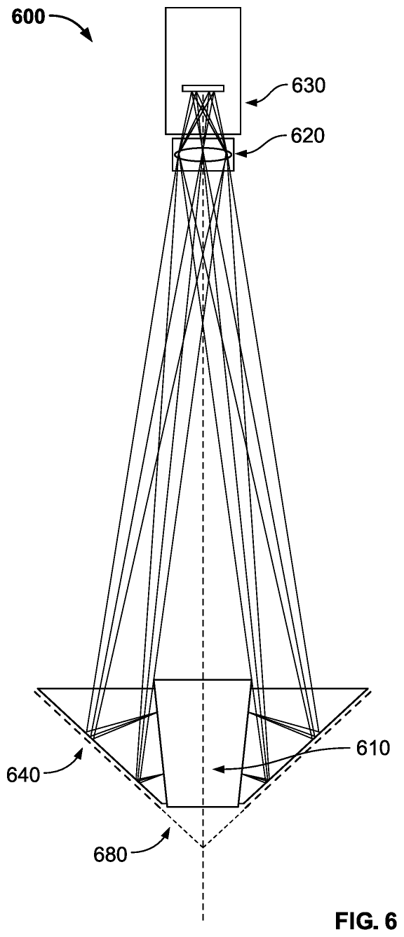

FIG. 6 illustrates a variation of the AMM illustrated in FIGS. 2 and 3. The system 600 illustrated in FIG. 6 has a lens/camera assembly 620/630. In the FIG. 6 variation, the extended apex 680 of the AMM 640 forms an angle of 96° which is a wider angle that provides a better reflected image of a tapered bottle 610.

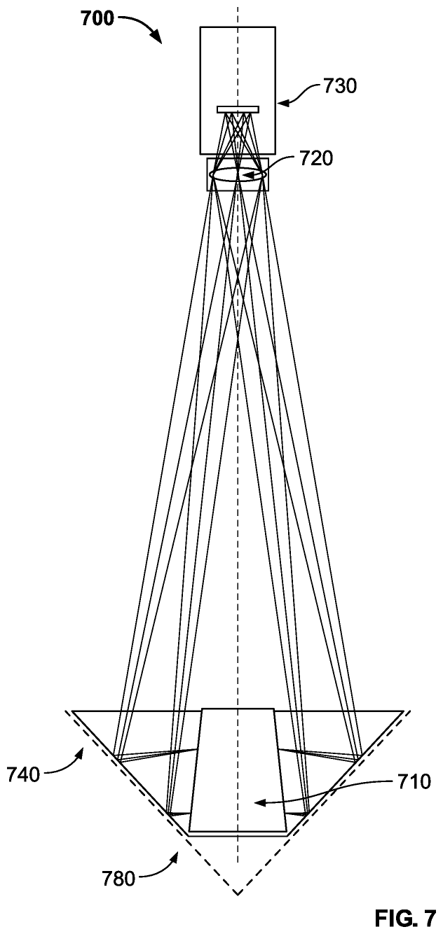

FIG. 7 illustrates a variation of the AMM illustrated in FIGS. 2 and 3. The system 700 illustrated in FIG. 7 has a lens/camera assembly 720/730. In the FIG. 7 variation, the extended apex 780 forms an angle of 84° which is a narrower angle that provides a better reflected image of a tapered bottle 710 in which the wider portion of the bottle is proximal to the apex 780.

In the embodiments of FIG. 2, FIG. 3, FIG. 6, and FIG. 7, uniform illuminations (not shown) are directed from light source(s) around or behind the imaging camera, the light directed downward toward the conical mirror. The conical mirror provides folding functions in the light path. In this way, the entire body of the bottle positioned at the center of the conical mirror is uniformly illuminated for imaging.

The examples of the AMM described herein that use the conical mirror provide 3D Path-Folding that provides an image of the entire body of the blood culture bottle. In an alternative embodiment, the imaging system may be replaced by a fluorescence detecting system. In this alternative configuration, the camera is replaced by a photo sensor. An emission filter is placed in front of the sensor. In this embodiment, the bottle is illuminated by excitation light having shorter wavelengths (for example, a narrow band of wavelengths centered at 560 nm. Accordingly, the emission filter placed in front of the sensor is a longpass filter with cut-on wavelength at 635, nm, for example. In this embodiment the bottle may be replaced by a test tube or a cuvette. The test tube or cuvette will be placed in the AMM just as the bottle is placed in the AMM as described herein. The test tube or cuvette will be illuminated by an excitation beam propagating upwards to the bottom of the test tube or cuvette.

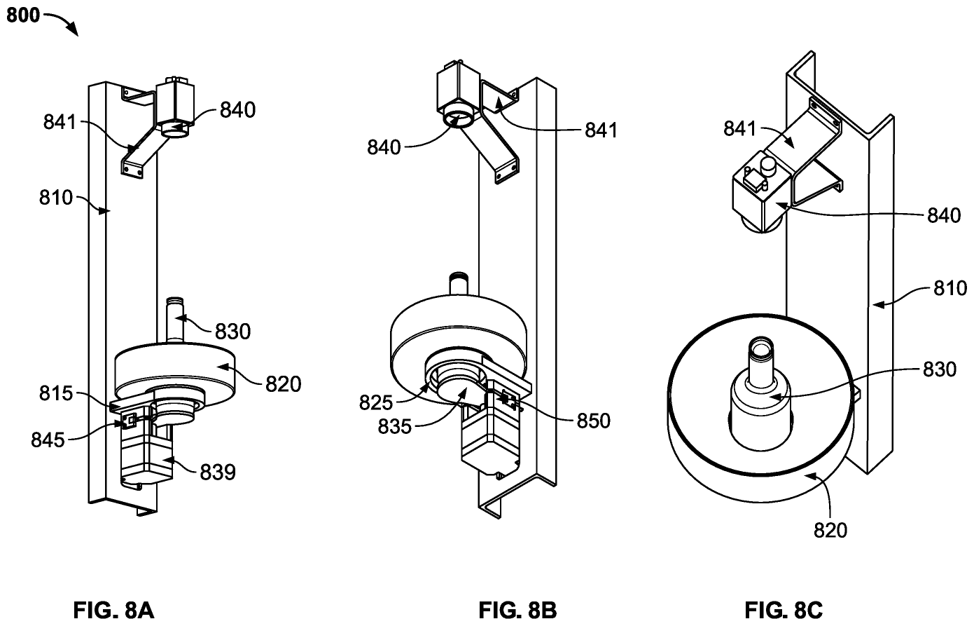

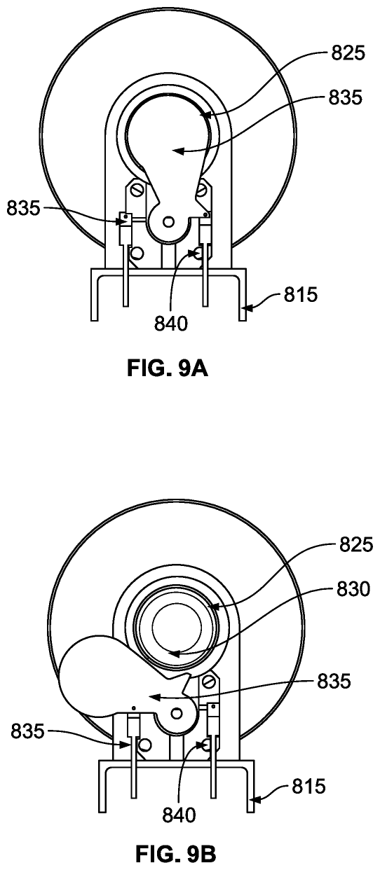

FIG. 8A-FIG. 8C illustrate an apparatus for receiving a bottle in a conical mirror for imaging. FIG. 8A is side perspective view of the apparatus 800 with support 810 for bracket 815 for holding the conical mirror 820 into which the bottle 830 is received for imaging. The bracket 815 has an opening 825 through which the bottle 830 will fit. A motor, 839, is fitted on the bracket 815 for moving the gate 835 from the closed position, which is illustrated in FIG. 8A-FIG. 8C and FIG. 9A, to the open position illustrated in FIG. 9B. Slotted optical switches, 845, 850 sense the open and closed positions of gate 835. The gate 835 is directly connected to a shaft (not shown) of the motor 840.

Positioned above the bracket 815 on support 810 is camera 840. Camera 840 is aimed downward to capture the image of a label (not shown) on the bottle 810. Camera 840 is affixed to support 810 by bracket 841. As described above, the conical mirror 820 allow for capture of an image of the entire label in one image, which is then processed by converting polar coordinates to cartesian coordinates, to yield an undistorted image of the label.

As illustrated in FIG. 9A, the bracket 815 has a gate 835 that supports the bottle 830 in the conical mirror 820 for imaging. When imaging is complete, the gate 835 is pivoted away as illustrated in FIG. 9B. Once the gate no longer covers the opening 825, the bottle 830 will drop from the bracket 815.

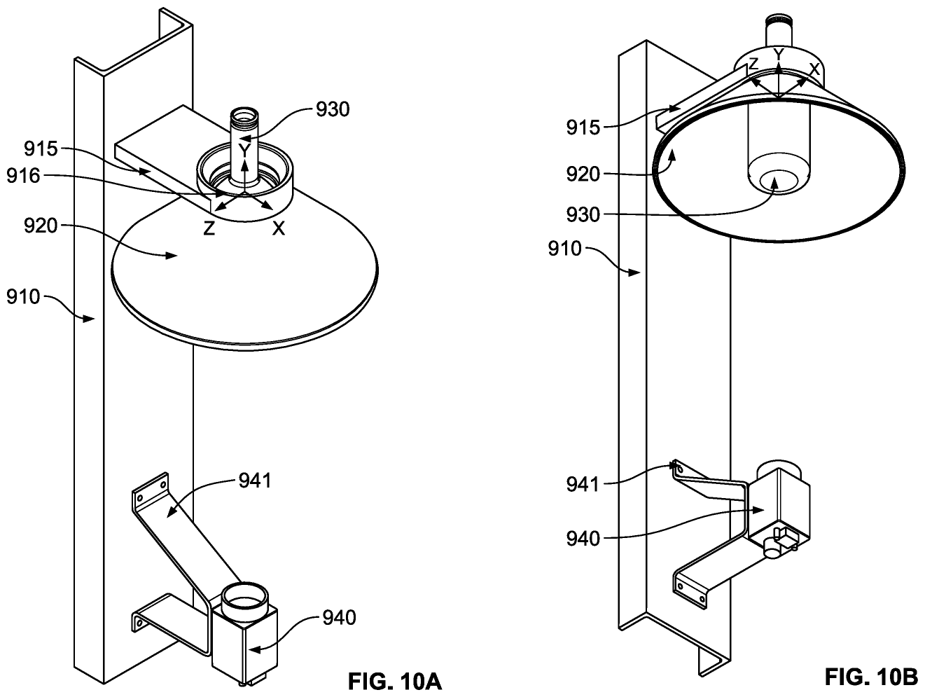

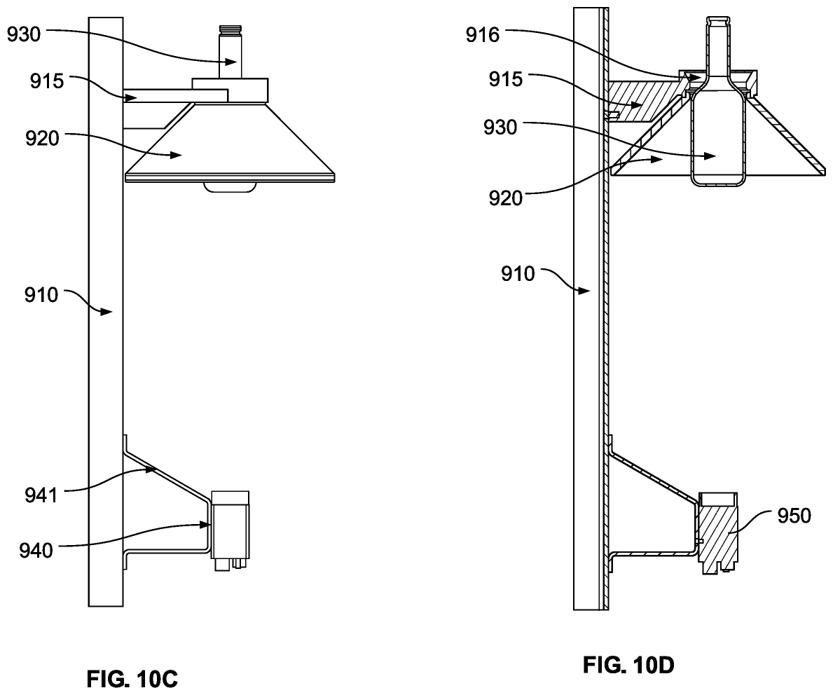

Referring to FIG. 10A-FIG. 10D, the conical mirror 920 is inverted and held by bracket 915 onto support 910. The bottle 930 is inserted through the opening 916 in bracket 915. In one aspect the bottle 930 is positioned into the conical mirror by a robotic arm (not shown) that holds the bottle 930 in place for imaging. One skilled in the art will appreciate that the bottle 930 is held in the conical mirror 920 by a number of different mechanical means. For example, the bracket 915 might be configured with a clamp that holds the bottle 930 in place for imaging. In another example, the bracket 915 might be configured with a tension ring that allows the bottle 930 to be passed through the ring with the application of sufficient force, but holds the bottle 930 in place when the force is no longer applied. The image is obtained by camera 940. Camera 940 is fixed to support 910 by bracket 941. As illustrated in FIG. 10D, the camera 940 is in communication with processor 950. Processor 950 receives a polar image of the label from the bottle that is the image of the label as reflected by the mirrored interior surface of conical mirror 920. The processor 950 is programmed with instructions to map the polar image of the label to cartesian coordinates using a polar transform. The image is transformed from an image of the label as reflected by the mirrored interior surface of conical mirror 920 using a polar transform.

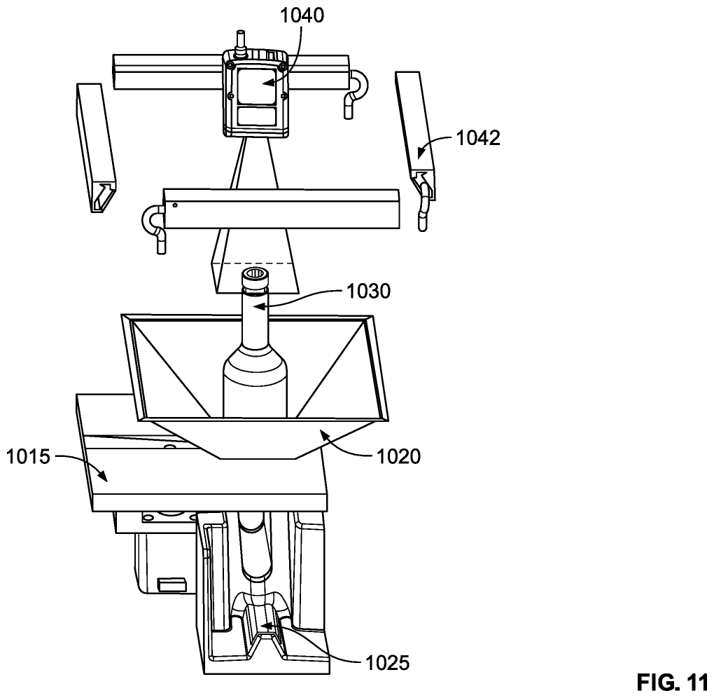

FIG. 11 is a schematic view of a pyramidal mirror 1020 that also provides a 360° image of a label on a cylindrical sample container 1030. The bottle 1030 is placed in the pyramidal mirror 1020 with the bottom resting on the narrower base of the pyramidal mirror. A camera 1040 is positioned above the pyramidal mirror 1020. The image may be formed by taking several images while rotating the mirror and stitching them together. Rails 1042, illustrated schematically, are provided for positioning the camera 1040 in x and y. Rotating motor 1025 rotates the cylindrical sample bottle 1030 as it sits in pyramidal mirror 1020. A trap door (not shown) is provided under the base 1015 of the pyramidal mirror 1020. When opened, the trap door permits the cylindrical sample container to drop into a chute (not shown) so that the cylindrical sample container 1030 may drop from the pyramidal mirror 1020 into the chute.

Disclosed herein are examples of systems that provide both the ability to obtain an image of the label on the cylindrical sample container and information about the contents of the sample container (e.g., blood volume, presence of foam, fill level, presence or absence of culture media in the neck of the cylindrical sample container). The systems and methods obtain this information yet maintain throughput speed so that a high number of cylindrical sample containers may be assessed quickly. Also required is an imaging environment that will permit the image information and the contents information to be accurately obtained. The systems are adaptable to different sizes and configurations of cylindrical sample containers, although all containers are envisioned to provide a cylindrical surface on which the label is placed.

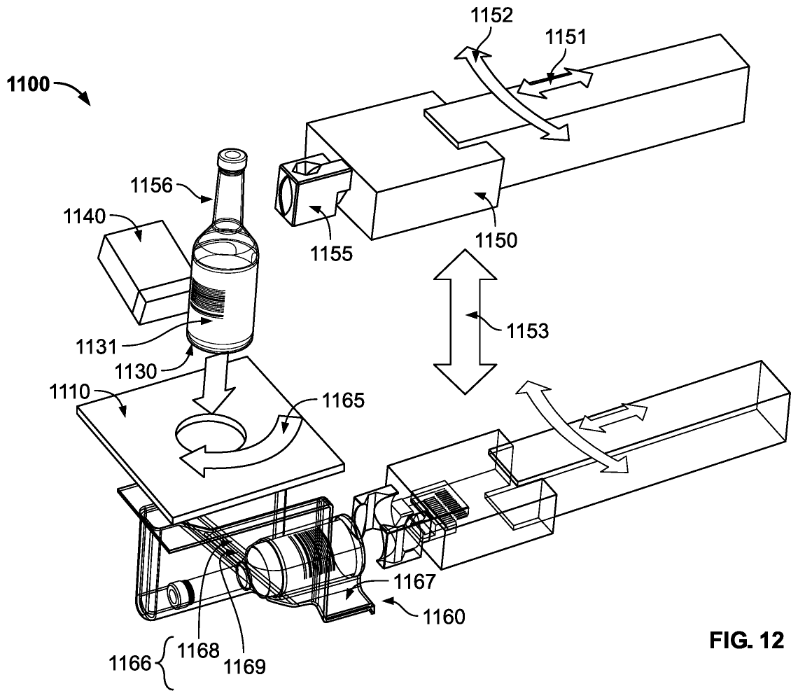

FIG. 12 is a schematic view of an imaging apparatus 1100. The apparatus has a platform 1110 on which the cylindrical sample container 1130 is placed. The apparatus 1100 also has a scanner 1140. A gripper arm 1150 with a clamp 1155 grips the neck 1156 of the cylindrical sample container 1130 and is used to place the cylindrical sample container 1130 onto the rotating gate 1165 of the platform 1110. The gripper arm 1150 is moveable in x (1151), y (1152), and z (1153) so that the gripper arm 1150 may be used to place the sample container 1130 on the rotating gate 1165 in the upright position and retrieve that sample container 1130 when the sample container is lying horizontally in the chute 1160. The chute 1160 receives the cylindrical sample container in the upright position and causes the cylindrical sample container to lay in the horizontal position. Therefore chute 1160 functions as a flip station to flip the cylindrical sample container from the upright position to the horizontal position. The gripper arm 1150 is rotatable so that the clamp 1155 may grip the cylindrical sample container 1130 when the cylindrical sample container is lying horizontally. In the apparatus described in FIG. 12, an image of the label 1131 is obtained as the cylindrical sample container is rotated by the rotating gate 1165. That image is then stitched together to form a complete image of the label 1131. Stitching images together to form a larger image is well known to one skilled in the art and is not described in detail herein.

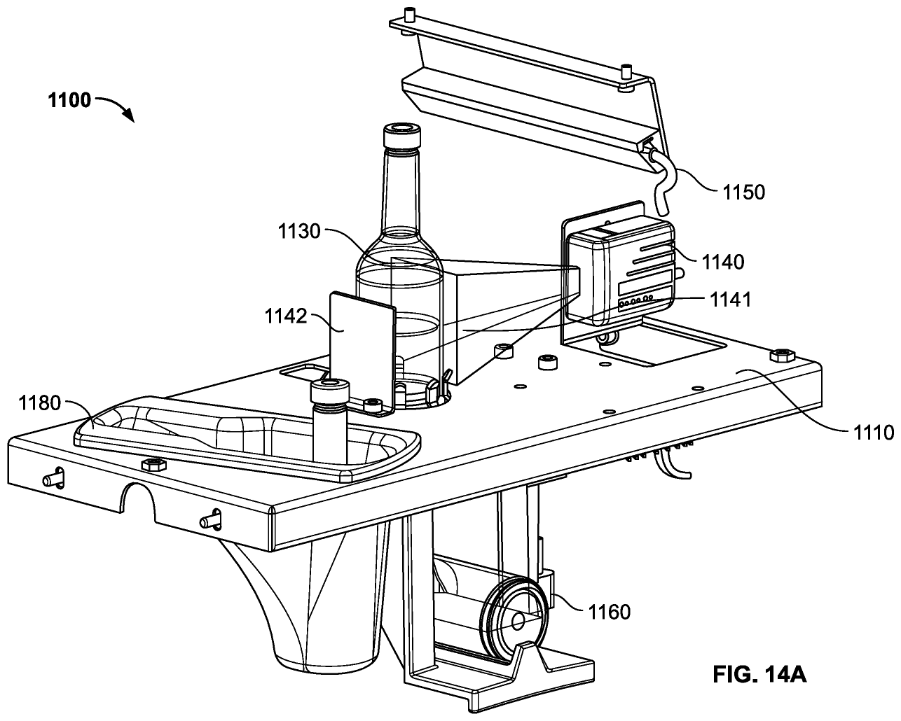

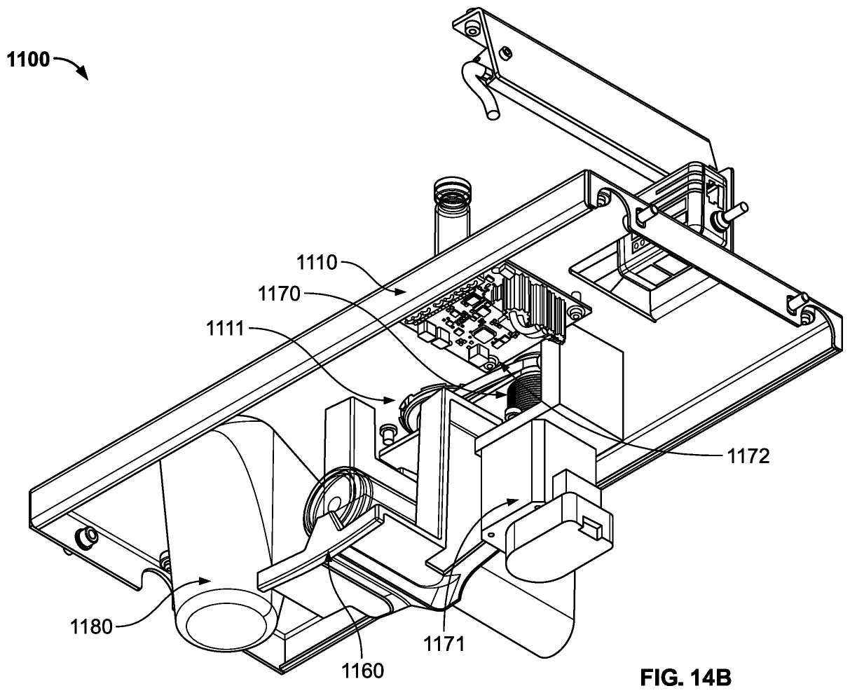

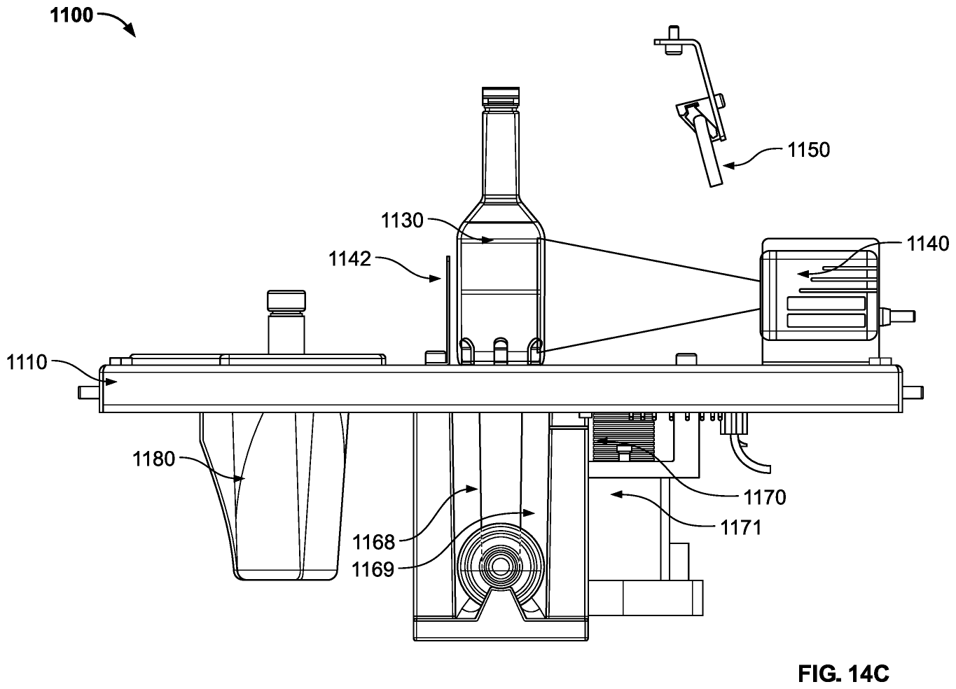

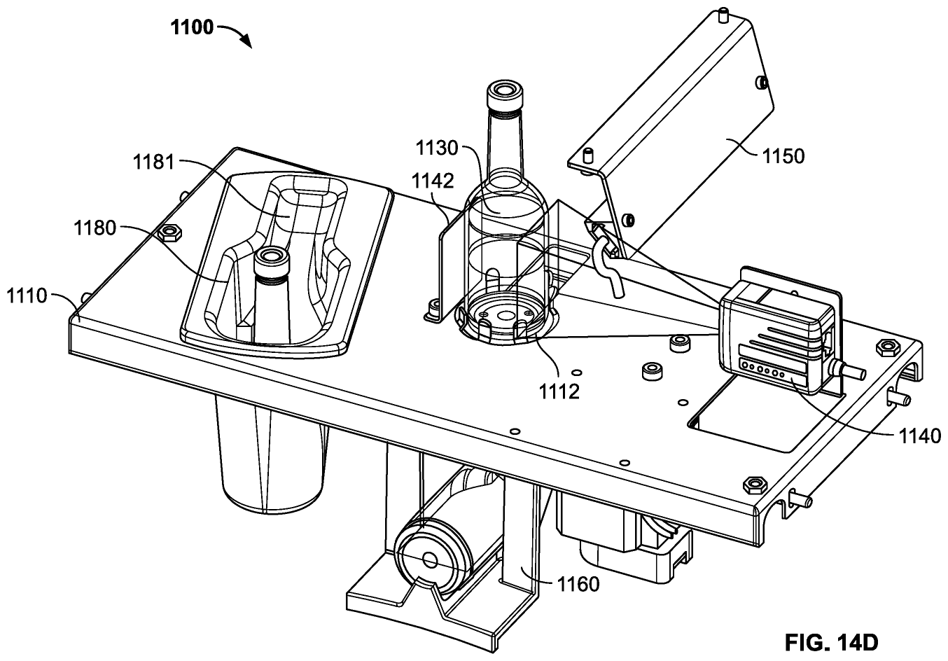

The rotating gate is rotated by a motor (not shown). Sensors inform the gripper arm 1150 when the clamp 1155 may release the cylindrical sample bottle 1130 on the rotating gate 1165. For imaging, the rotating platform 1110 (the rotating gate 1165 is located below the surface of the main portion of the platform 1110) rotates in one direction (either clockwise or counter clockwise). After the imaging apparatus 1100 has obtained an image of the entire label 1131 and has also obtained image information from which the presence or absence of foam, fill level and other information regarding the contents of the cylindrical sample container, imaging apparatus (e.g., camera, scanner, lights, etc.) are turned off. The rotating gate 1165 may also be actuated out of alignment with the chute 1160. When the rotating gate 1165 is aligned with the chute 1160, the cylindrical sample container does not slip through the chute when the bottle is placed on the rotating gate 1165 for imaging. The complete image may be formed by taking several images before and after rotating the bottle by about 45 degrees and then stitching those images together to provide an image of the complete bottle. After imaging, the rotating platform 1110 rotates in the opposite direction until the gate 1165 is actuated out of alignment with the opening for the chute 1160. This allows the cylindrical sample container 1130 to slip through the opening the chute 1160, which has a ramp 1166 and a platform 1167. The cylindrical sample container 1130 eases down ramp 1166 and comes to rest horizontally on platform 1167, from where it is retrieved by the clamp 1155 of gripper arm 1150. In this regard the ramp 1166 has tracks 1168, 1169 which are spaced apart so that, as the cylindrical sample container 1130 eases down the ramp 1166, the neck of the cylindrical sample container 1130 fits between tracks 1168, 1169, allowing the cylindrical sample container 1130 to lie flat. Tracks 1168 and 1169 are more readily observed in FIG. 14 C.

Not shown are a calibration plate that is disposed on the end of the platform 1110 opposite the scanner 1140. The calibration plate may be used to calibrate the scanner 1140 to ensure that, when the cylindrical sample container 1130 is placed on the rotating gate 1165, it will be in the correct field of view for the scanner. The rotating gate 1165 is configured to provide a stable surface on which to set the cylindrical sample container 1130 for imaging. Since sterilizing the cylindrical sample containers prior to use may introduce deformities or irregularities in the bottom surface of the cylindrical sample containers 1130, the rotating gate 1165 may be provided with recessed portion that will allow the perimeter of the bottom of the cylindrical sample container to seat securely on the rotating gate 1165 yet provides a clearance between the interior of the bottom surface of the cylindrical container and the surface of the cylindrical sample container 1130 so that any surface deformities do not cause the cylindrical sample container to seat in an unstable manner.

Alternatives structures to the rotating gate include rubber drive wheels that are adject the cylindrical sample container or rotating grippers such as those used to screw on or screw off caps automatically. If such rotating mechanisms are used, the system is provided with a trap door or other mechanism to allow the cylindrical sample container to advance into the chute when the imaging is complete.

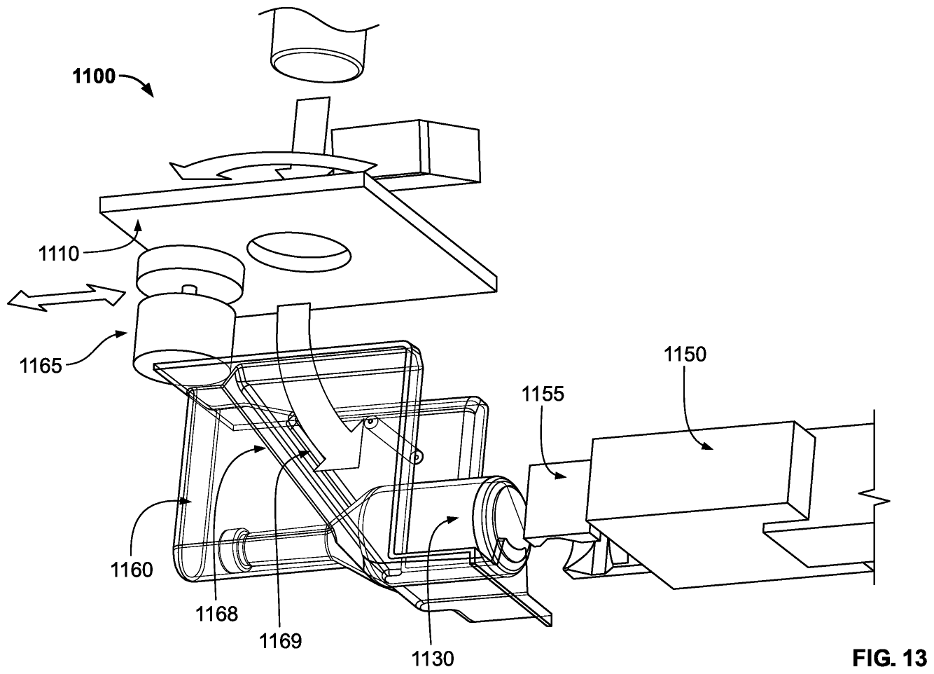

FIG. 13 is a bottom view of the imaging apparatus 1100 of FIG. 12. IN FIG. 13 the rotating gate 1165 is illustrated out of alignment with chute 1160. After the cylindrical sample container 1130 has traveled down chute 1160, it rests in a horizontal position with its neck disposed between tracks 1168, 1169. The clamp 1155 of the gripper arm 1150 rotates to grip the bottom of cylindrical sample container 1130 to remove it from the chute.

FIGS. 14A-FIG. 14D illustrate an alternative embodiment system 1100 of FIG. 12 in which platform 1110 has a rotating platform 1111 mounted beneath it. FIG. 14A is a perspective view from above the system 1100. FIG. 14B is an upward perspective view of the system 1100. FIG. 14C is a side view of the system 1100. FIG. 14D is a downward perspective view of the system 1100. The rotating platform 1111 is driven by shaft 1170 that is rotated by a motor 1171 from which the shaft 1170 extends. A belt 1172 couples the shaft 1170 to the rotating platform 1111, causing the rotation of the rotating platform 1111. After the camera 1140 obtains an image of the cylindrical sample container 1130, the rotation of the shaft 1170 is reversed, and, when the rotating platform rotates in the opposite direction, the rotating platform 1111 is pivoted away, allowing the cylindrical sample container 1130 to slide into chute 1160 through opening 1112 in platform 1100.

The embodiment of FIGS. 14A-14D has a holding station 1180, for the cylindrical sample container 1130. The holding station 1180 has a ramp structure 1181 so that the cylindrical sample container 1130 will sit in an upright position as long as it placed bottom first into the holding station 1180. The robotic arm 1150 is used to bring the cylindrical sample container 1130 into the holding station 1180. The robotic arm 1150 also moves the cylindrical sample container 1130 to the imaging location 1141 and places it therein, and retrieves the cylindrical sample container 1130 from the chute 1160. A plate 1142 is set behind the imaging location 1141 to provide a static background for the image. The cylindrical sample container 1130 is rotated a predetermined number of degrees (e.g., 20 degrees, 30 degrees, etc.) and the images at each rotation increment are then stitched together to obtain an entire image of the label.

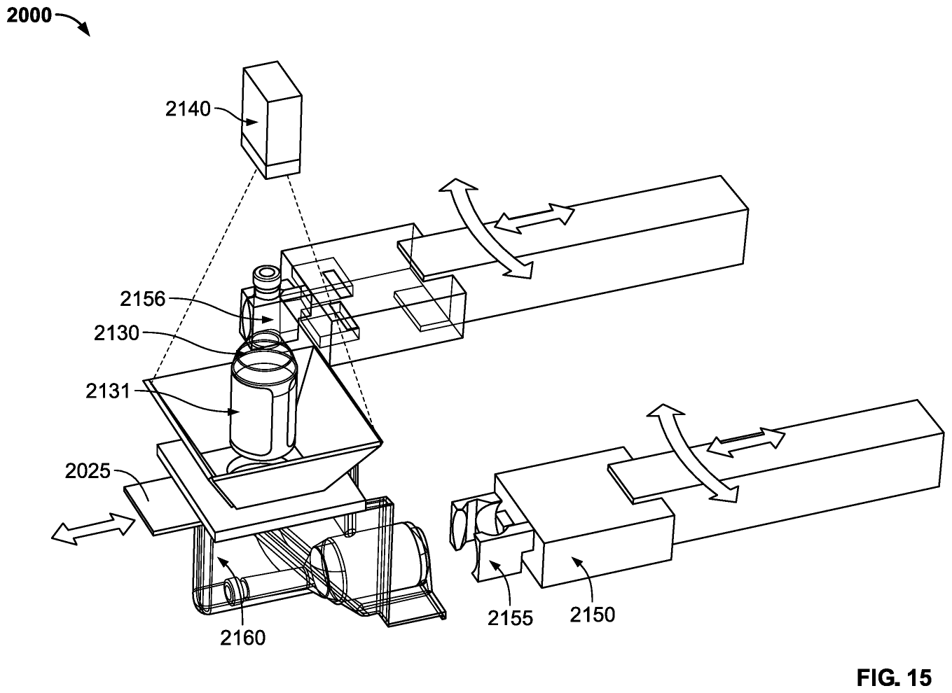

FIG. 15 is an alternative embodiment of FIG. 12, but one in which the cylindrical sample container is not rotated. In this embodiment, the system 2000 has a pyramidal mirror 2020 such as the one illustrated and described in FIG. 11 for which the complete image can be formed by taking several images before and after rotating the bottle about 45 degrees between images and then stitching the images together. The system has a trap door 2025 that slides horizontally. When the trap door 2025 is advanced inward, it holds the cylindrical sample container 2130 in place for imaging by scanner 2140. The image that is captured is of the entire label 2131. A gripper arm 2150 with a clamp 2155 grips the neck 2156 of the cylindrical sample container 2130 and is used to place the cylindrical sample container 2130 into the pyramidal mirror 2020 for imaging. The gripper arm 2150 is moveable in x, y, and z so that the gripper arm 2150 may be used to place the cylindrical sample container 2130 in the pyramidal mirror 2020 in the upright position and retrieve the cylindrical sample container 2130 when the cylindrical sample container is lying horizontally in the chute 2160. When the trap door 2025 is advanced outward, the cylindrical sample container 2130 falls through the chute 2160 and is removed by the gripper arm 2150. Alternative embodiments deploy other types of doors for allowing the cylindrical sample container to descend into the chute 2160. Examples of suitable alternative doors include drop away doors, sliding doors or a retracting pin.

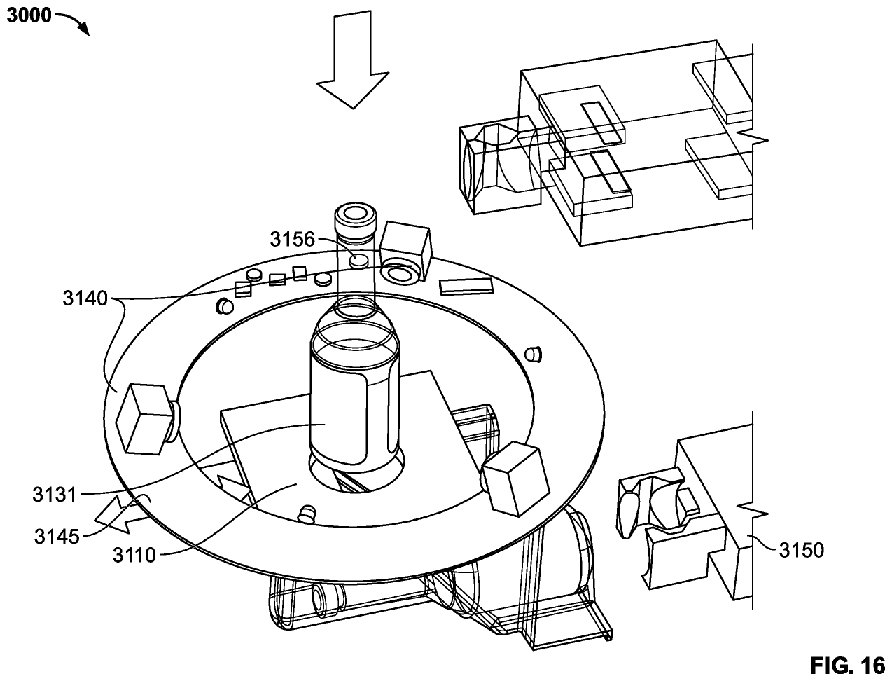

FIG. 16 illustrates an alternative system 3000 in which multiple cameras 3140 are used to obtain images of the label 3131 on the cylindrical sample container 3130. Obtaining an image with multiple cameras is a well-known technique for assembling a “flat” image from a cylindrical object, as each image is only a segment of the curved object. Stitching such images together is also well-known and not described in detail herein. The system 3000 has a platform 3110 with a trap door 3025. The trap door 3025 is closed and the cylindrical sample container is held on the platform 3110 for imaging. As illustrated, the cameras 3140 are mounted on a ring-shaped printed circuit board 3145. As described above, a gripper arm 3150 is used to grip the neck 3156 of the cylindrical sample container 3130 and place it in the imaging apparatus. After imaging, the trap door 3025 is actuated and the cylindrical sample container 3130 falls through the chute 3160 and is removed by the gripper arm 3150.

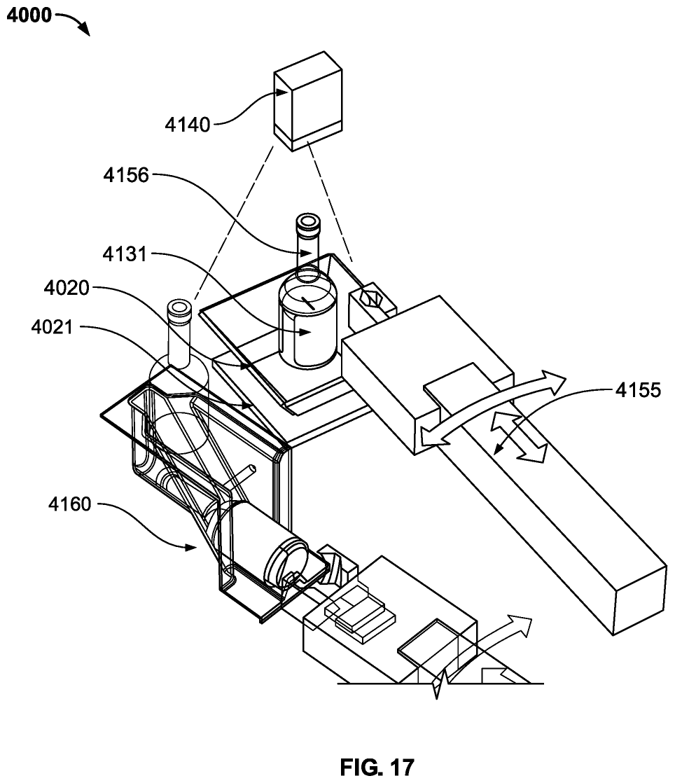

FIG. 17 illustrates a system 4000 that does not have a trap door. In this embodiment, the gripper arm 4150 is used to place and remove the cylindrical sample container 4130 from the pyramidal mirror 4020 which has no opening in its base 4021. The scanner 4140 is used to obtain a single image of the entire expanse of label 4131. After imaging, in this embodiment, should the user seek to have the cylindrical sample container gripped by the base rather than the neck, the gripper arm 4150 will remove the cylindrical sample container 4130 from the pyramidal mirror 4020 and place it in the chute 4160 wherein it will slide to a horizontal position as described above, after which the gripper arm 4150 will remove the cylindrical sample container from the chute 4160 by gripping the base of the cylindrical sample container 4130.

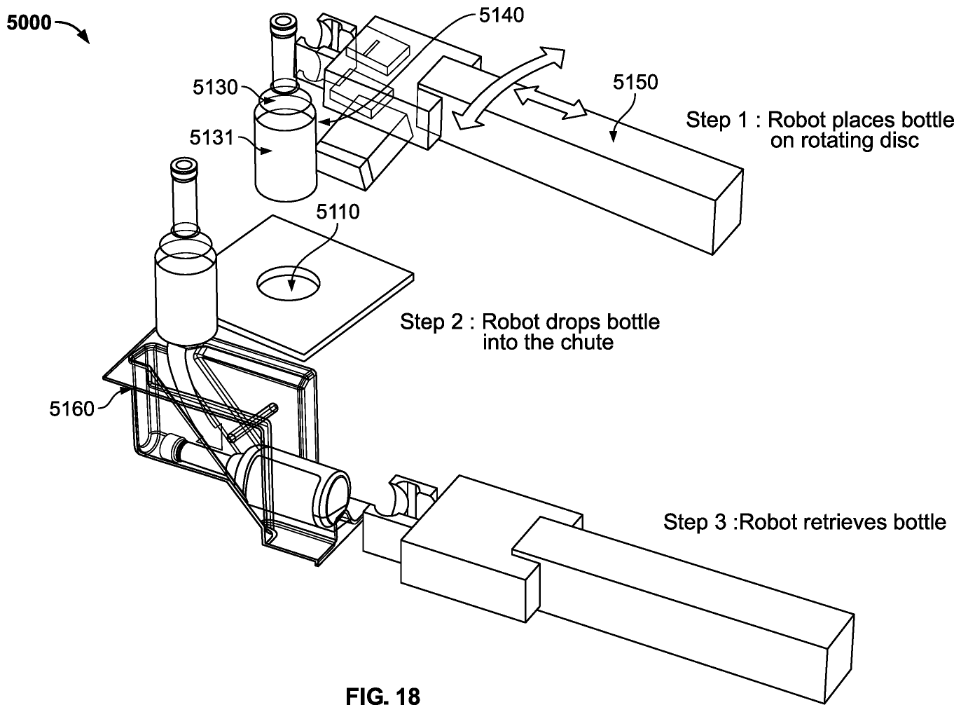

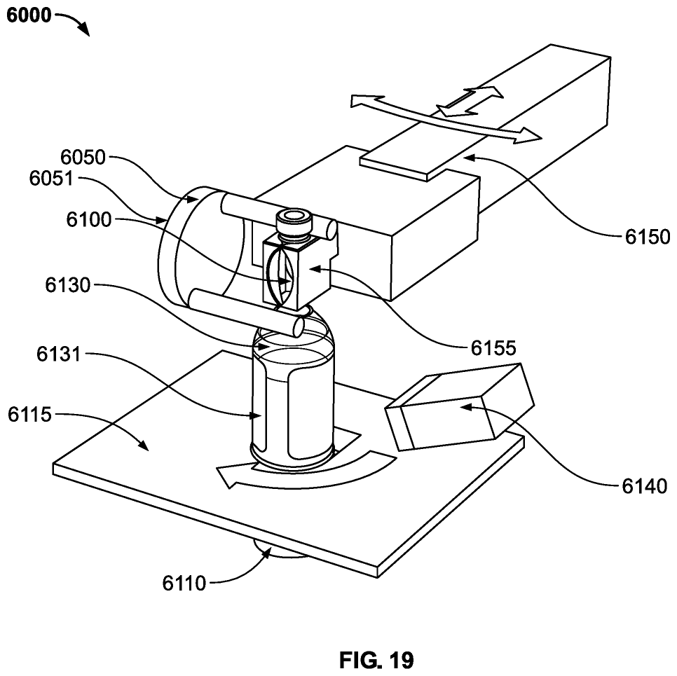

FIG. 18 is a system 5000 such as is illustrated in FIG. 12 but wherein the gripper arm 5150 moves the cylindrical sample container 5130 to the imaging position and to the chute 5160 that flips the cylindrical sample container from the upright to the horizontal position. The gripper arm 5150 has the scanner 5130 mounted thereon. Once the gripper arm 5150 places the cylindrical sample container 5130 on to the rotating platform 5110, the gripper arm then advances to align the scanner 5140 with the cylindrical sample container 5130 to obtain an image of the label 5131 as the rotating platform 5110 rotates the cylindrical sample container 5130. After the image of the cylindrical sample container is obtained, the gripper arm 5150 then moves the cylindrical sample container to the chute 5160. When placed in the chute 5160 the cylindrical sample container flips from the vertical position in which it is placed to the horizontal position, where it is retrieved by the gripper arm 5150 by grasping the bottom of the cylindrical sample container 5130.

FIG. 19 is a system 6000 that does not use a chute to rotate the cylindrical sample container from the upright position to the horizontal position. System 6000 deploys a tilting gripper 6050 that grips the neck 6100 of the cylindrical sample container 6130. The system 6000 uses the rotating platform 6110, placed underneath platform 6115 to ensure that images of the entire label 6131 are captured by the scanner 6140. After an image of the cylindrical sample container 6130 is captured by the scanner 6140, the end effector 6155 of the gripper arm 6150 rotates and sets the tilting gripper 6050 such that the flat surface 6051 of the tilting gripper rests on the platform 6115. The gripper arm then releases the neck 6100 of the cylindrical sample container 6130. The cylindrical sample container is then held in the horizontal position by the tilting gripper 6050 resting on platform 6115. The gripper arm 6150 then rotates and advances end effector 6155 in position to grasp the bottom of the cylindrical sample container 6130, which is being held in the horizontal position. The end effector 6155 then grasps the bottom of the cylindrical sample container 6130 and conveys the cylindrical sample container 6130 away from the platform 6100. The tilting gripper is not conveyed away with the cylindrical sample container 6130.

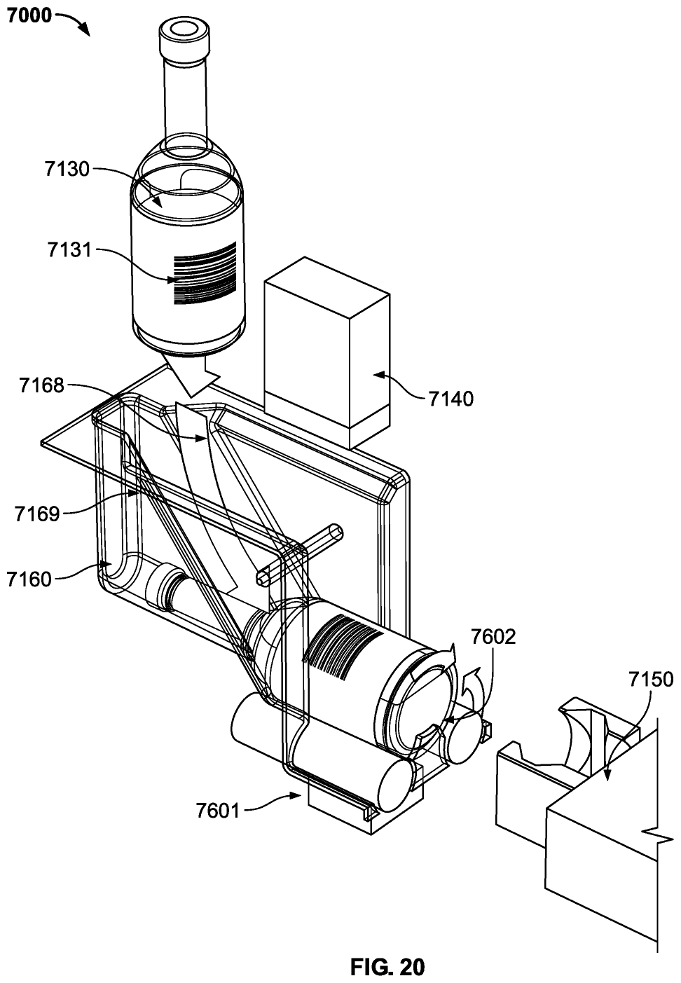

FIG. 20 illustrates a system 7000 that deploys the chute 7160 to rotate the cylindrical sample container 7130 as it lays horizontally in the chute 7160. As described above, the gripper arm 7150 holds the cylindrical sample container in a vertical orientation. The gripper arm 7150 places the cylindrical sample container 7130 in the chute where it slides down ramp 7166 along tracks 7168 and 7169. The neck 7100 of the cylindrical sample container 7130 fits between tracks 7168, 7169. The chute 7160 has rollers 7601 and 7602. The rollers may be used to cause the cylindrical sample container 7130 to rotate. With the scanner 7140 placed over the rotating cylindrical sample container, an image of the label 7131 is obtained. However, when the cylindrical sample container 7130 is in the horizontal position, the meniscus of the inoculated culture in the neck 7100 of the cylindrical sample container 7130 cannot be observed. Therefore, in this system, the volume of the sample (e.g., blood) added to the sample cannot be ascertained by observing the cylindrical sample container in its horizontal position. After an image of the label 7131 on the cylindrical sample container is obtained, the gripper arm 7150 grabs the bottom of the cylindrical sample container 7130 to remove it from chute 7160.

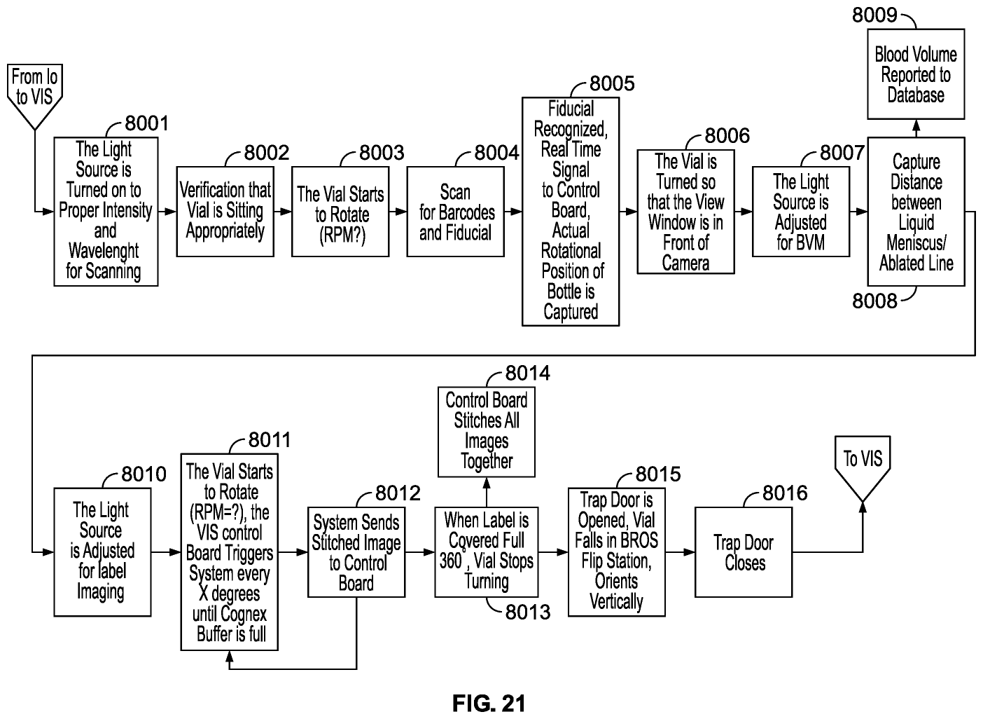

FIG. 21 is a flow chart for the positioning of the cylindrical sample container for imaging. In step 8001, the light source for the scanner is turned on and tuned so the correct intensity and wavelength for scanning. This step is controlled by software. In step 8002, sensors verify that the cylindrical sample container is in the correct position. In those embodiments where the cylindrical sample container is rotated, rotation of the bottle is commenced in step 8003. The scanner then scans the bar code and any fiducial marks on the cylindrical sample container in step 8004. In step 8005, when the fiducial is recognized, the position of the cylindrical sample container is captured by the system. In step 8006, the cylindrical sample container is rotated so that the view window (i.e., a portion of the cylindrical sample container that is not covered by the label) is disposed in front of the scanner/camera to determine the liquid level in the cylindrical sample container. In step 8007, the light source is adjusted for blood volume measurements (BVM). In step 8008 the system captures a distance between the liquid meniscus in the cylindrical sample container and a line etched on the cylindrical sample container (for volume determination). The ablation line is etched at a custom height on the bottle during manufacturing denoting the intended fill level of the patient blood at bedside. Typical fill is 8-10 ml for adults and 3 ml for pediatrics using special peds bottle. Each media type has a published expected fill volume, which is used in computing amount of user overfill or underfill. The volume of the patient blood in the sample container is determined using the difference in height between the blood line and the ablation line. By knowing the volume characteristics of the cylindrical sample container, the amount of patient blood fill is calculated.

In step 8009 the blood volume is reported to the data base. In step 8010 the light source is adjusted (e.g., from blue to red or white) to obtain an image of the label. In step 8011, the cylindrical sample container is rotated at the set speed. An image is captured after a preset number of degrees (e.g., 20 degrees) of rotation until a full series of images of the entire label is obtained. In step 8012, the images are stitched together to form a full image of the label. The stitched image information is fed back to the rotation controller, which continues to rotate the cylindrical sample container until the buffer that receives the image information is full. In step 8013, when the cylindrical sample container has rotated a full 360 degrees, the rotation is stopped. In step 8014 all of the label images are stitched together. In those systems where a trap door is provided to release the cylindrical sample container into the chute, the trap door is opened in step 8015. In step 8016, the trap door closes. In those systems where the chute flips from vertical to horizontal, the cylindrical sample container is retrieved in the horizontal position.

As utilized herein, the terms “approximately,” “about,” “substantially” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described without restricting the scope of these features to the precise numerical ranges provided. Accordingly, these terms should be interpreted as indicating that insubstantial or inconsequential modifications or alterations of the subject matter described and are considered to be within the scope of the disclosure.

From the foregoing and with reference to the various figure drawings, those skilled in the art will appreciate that certain modifications may also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. A system for obtaining an image of a cylindrical object comprising:
a robotic conveying arm comprising an end effector with a clamp, wherein the clamp is adjustable to grip cylindrical objects with different diameters and wherein the robotic conveying arm is movable in x, y, and z;
a platform for receiving a cylindrical sample container, wherein the sample container is placed on the platform by the robotic conveying arm;
a camera positioned to obtain an image of the cylindrical sample container when placed on the platform, wherein the cylindrical sample container is placed on the platform in a vertical orientation and wherein the cylindrical sample container carries a label and wherein the camera is placed to obtain one or more images of the label carried by the cylindrical sample container;
wherein the system constructs a two-dimensional image of the label from the one or more images of the label obtained by the camera; and
wherein, after an image of the label is obtained, the cylindrical sample container is removed from the system by the robotic conveying arm.

2. The system of claim 1, wherein the cylindrical sample container is a blood culture bottle.

3. The system of claim 1, further comprising a rotating platform on which the cylindrical sample container is place.

4. The system of claim 3, wherein the camera obtains an image of the label in a single frame.

5. The system of claim 4, wherein the camera is in communication with a processor.

6. The system of claim 5, wherein the processor is programmed to apply a polar transform to the image received from the camera.

7. The system of claim 6, wherein the processor outputs a transformed image from applying the polar transform.

8. The system of claim 3, wherein the rotating platform rotates the cylindrical sample container by 360 degrees and the camera obtains an image of the label in n images, each image being taken as the cylindrical sample container rotates a predetermined portion of the 360 degrees.

9. The system of claim 8, wherein the predetermined portion of the 360 degrees is 45 degrees.

10. The system of claim 9, wherein the n images are stitched together to obtain an image of the label.

11. The system of claim 1, further comprising a chute disposed beneath the platform.

12. The system of claim 1, wherein a gate is placed on the platform, wherein the gate is operable from a first position in which the cylindrical sample container is supported on the platform to a second position where the cylindrical sample container is allowed to pass through an opening in the platform that results when the gate is moved to the second position.

13. The system of claim 1, wherein the chute receives the cylindrical sample container in an approximately vertical orientation and eases the cylindrical sample container into a an approximately horizontal orientation.

14. The system of claim 3, wherein the cylindrical sample container is a blood culture bottle.

15. The system of claim 11, wherein the chute comprises a sloped ramp that guides the blood culture bottle to the approximately horizontal orientation.

16. The system of claim 15, wherein the ramp is a pair of sloped tracks with an opening therebetween, wherein a neck of the blood culture bottle fits between the sloped tracks as the cylindrical sample container pivots from the approximately vertical orientation to the approximately horizontal orientation.

17. The system of claim 11, wherein the end effector of the robotic conveying arm grasps a bottom of the cylindrical sample container and wherein the robotic conveying arm conveys the cylindrical sample container from the chute when the cylindrical sample container is in the horizontal position.

18. The system of claim 1, further comprising an auxiliary mirror module comprising an angled mirrored interior surface interposed between the cylindrical sample container and the camera, wherein the camera is positioned such that it can capture an image of the cylindrical sample container when placed on the platform.

19. The system of claim 18, wherein the angled mirrored surface comprises two angled side mirrors wherein the side of the mirror facing the cylindrical object reflective, wherein the mirrored surfaces of the two angled side mirrors are configured to direct light from the reflective surfaces to reflective surfaces on a central angled mirror, wherein the reflective surfaces on the central angled mirror are configured to direct light toward the camera.

20. The system of claim 19, wherein the angle of the reflective surface of a first angled side mirror is about +45 degrees with respect to an axis from the cylindrical object to the camera and lens assembly and the angle of the reflective surface of a second angled side mirror is about-45 degrees with respect to an axis from the cylindrical object to the camera.

21. The system of claim 20, wherein the central angled mirror comprises a first angled reflective surface and a second angled reflective surface wherein the first and second angled reflective surfaces are at about +45 degrees and about −45 degrees, respectively.

22. The system of claim 19, wherein the angle of the reflective surface of a first angled side mirror is about +37 degrees with respect to an axis from the cylindrical object to the camera and lens assembly and the angle of the reflective surface of a second angled side mirror is about −37 degrees with respect to an axis from the cylindrical object to the camera.

23. The system of claim 22, wherein the central angled mirror comprises a first angled reflective surface and a second angled reflective surface wherein the first and second angled reflective surfaces are at about +45 degrees and about −45 degrees, respectively.

24. The system of claim 19, wherein the angle of the reflective surface of a first angled side mirror is about +35 degrees with respect to an axis from the cylindrical object to the camera and lens assembly and the angle of the reflective surface of a second angled side mirror is about −35 degrees with respect to an axis from the cylindrical object to the camera.

25. The system of claim 24, wherein the central angled mirror comprises a first angled reflective surface and a second angled reflective surface wherein the first and second angled reflective surfaces are at about +45 degrees and about −45 degrees, respectively.
